(19) United States
(12) Patent Application Publication
(10) Pub. No.: US 2026/0108304 A1
(43) Pub. Date: Apr. 23, 2026

(54) IMPLANT PLANNING SOFTWARE

(71) Applicant: Massimo AFFLITTO, Torino (IT)

(72) Inventor: Massimo AFFLITTO, Torino (IT)

(21) Appl. No.: 18/920,427

(22) Filed: Oct. 18, 2024

(51) Int. Cl. A61B 34/10; A61C 13/00; G16H 20/40

(52) U.S. Cl. CPC A61B 34/10; A61C 13/0004; G16H 20/40

(57) ABSTRACT

A modified digital library and an algorithm able to make six functions for the improvement of most implant planning software used for the design of the custom surgical guide are provided. The prior art implant planning software available on the market, improved by the modified digital library and algorithm presented here, is essential to guide the digital design of both the custom surgical guide and the CAD-CAM cemented prosthesis onto straight or angled prefabricated abutments, through preliminary determination of abutment position with respect to an appropriate system of reference planes in correspondence with digital crown design. Furthermore, in a future perspective, the improved implant planning software presented here could be usefully employed in robot-assisted dental implant surgery.

### FIELD OF THE INVENTION

The present invention relates to method for optimizing digital design of 3D placement of implant osteotomy, with the emphasis placed on the position of prefabricated abutment as a driving element in the implant position.

### BACKGROUND OF THE INVENTION

Dental implants (hereinafter simply referred to as the implants) are designed for placement into the alveolar or basal bone of the mandible or maxilla and are used to retain fixed prosthesis (which are either screw-retained or cement-retained, i.e. crowns and bridges) or removable prosthesis. More precisely, an abutment is connected to the implant to allows the prosthesis to be connected to the implant. There are four basic types of prefabricated abutments for fixed prosthesis, of which two for cemented prosthesis (see FIGS. 1A-B) and two for screwed prosthesis (see FIGS. 1C-D). As a general rule, a decisive factor in the proper use of an angled prefabricated abutment is the alignment of implant's hex connection (female hex) during implant insertion. In particular, during implant insertion, one side of the hexagon of the implant connection must be aligned by eye to one side of imaginary occlusal square overhanging each crown that will be fixed later on the implant, facially (for the subsequent correction of the facial or lingual implant angulation, FIG. 2B) or mesially (for the subsequent correction of the mesial or distal implant angulation, FIG. 2C). Each imaginary occlusal square graphically illustrates the four side faces of the respective digitally designed crown (see FIG. 2A). The dotted circles present in FIGS. 1B-C highlight the position of the angled prefabricated abutment's hex connection (male hex) to take into account during implant insertion.

Prior to the placement of implant, osteotomy site must be prepared. Usually, multiple drills are used to sequentially widen the osteotomy to the matching diameter of the implant being placed. The osteotomy site preparation can be accomplished freehand or increasingly via a “custom surgical guide” also known as “CAD-CAM surgical guide” (FIG. 3A). Furthermore, although we are at the dawn of robotic implant surgery, this is a third technique to keep in mind.

In short, while for freehand osteotomy are sufficient 2D radiographs (i.e. a periapical or a panoramic images), guided osteotomy, via a custom surgical guide, requires CBCT images (3D radiograph of the anatomy of the maxilla or the mandible) in combination with the implant planning software (FIG. 3B). The guided osteotomy allows the highest level of precision and control because the implant position is dictated via a 3D evaluation of the anatomy. The 3D placement of osteotomy (i.e. the position, angulation, and depth of the osteotomy) is dictated by the “sleeve”, also known as guided tube, which is incorporated into the CAD-CAM surgical guide, restricting any variation by the implant surgeon, therefore osteotomy drills are used through each sleeve of the CAD-CAM surgical guide. CAD-CAM surgical guides are computer-generated drilling guides (CAD procedure, see FIG. 4) that are fabricated through the process of 3D printing (CAM procedure, not shown).

The prior art implant planning software permit digital placement of the implants based on the anatomy of the patient (FIG. 3B), the designing of the custom surgical guide (FIG. 3A) and also allows the clinician to select prefabricated abutments (see FIGS. 1A-D) consistent with the crown/bridge design. The implant planning software also provides a digital library containing both the commercially available prefabricated abutments and the commercially available implants. Furthermore, the implant planning software allows you to pair an abutment and an implant both selected from a digital library, to form the abutment-implant unit.

As a first step in CAD designing any custom surgical guide (FIG. 4C), the crown/bridge design (FIG. 4A) has to be stablished (the so-called digital waxing). Implant brand, model, length and diameter, as well as the related straight or angled prefabricated abutments, can be selected from software library. Therefore, once digital crown design has been finished, the CAD procedure first determines the implant position within bone (corresponding characteristic to the placement of implant osteotomy, see FIG. 4B) and only then the position and angulation of prefabricated abutment. Placement a tooth provided by the library and adapting it to the desired clinical situation comes prior to implant placement, as the intention of prior art protocol is to deliver an implant that is prosthetically driven. However, this technique only partially follows the sacrosanct descending principle of “prosthetically driven 3D implant placement”, i.e. proceeds from the crown directly to the implant and only later to the abutment. This procedure presents at least four different drawbacks, all due to the absence of landmarks in correspondence with digital crown design.

One drawback of this technique is that having to determine the implant insertion axis without reference planes, it is a time-consuming procedure and operator-dependent.

Another drawback of this technique is that an off-center placement of implant's hex connection (the implant's hex connection is the upper part of the implant that mates with an abutment's hex connection, see FIG. 5) with respect to the occlusal face of the crown, of frequent finding, is responsible for a non-uniform thickness in the crown, often makes it necessary to resort to customized abutments (FIG. 6B), more expensive than prefabricated ones.

Yet another drawback of this technique is that at most it can create the parallelism of the implants (FIG. 6A) but not the parallelism of the prefabricated abutments, which is much more useful in cemented bridges. Yet another drawback of this technique is that an undesirable implant axis can cause an unwanted choice of inclination of prefabricated abutment or an implant placed too far from the adjacent tooth during the planning process (FIG. 7).

### SUMMARY OF THE INVENTION

In view of the foregoing, a need exists for an improved implant planning software that produces operative results that are sufficiently predictable, repeatable, non operator-dependent, more cost-effective and/or accurate: therefore, this invention totally follows the sacrosanct descending principle of “prosthetically driven 3D implant placement”, i.e. proceeds from the digital crown design to the position of prefabricated abutment and only later to the implant position.

Yet another aspect of the present invention relates to an employment of the improved implant planning software in robot-assisted dental implant surgery.

A first improvement of the prior art implant planning software provided by the present invention concerns the introduction of modified digital library in which all prefabricated abutments available on the market are equipped with two landmarks, that are the plane of symmetry and the longitudinal axis, where plane of symmetry divides abutment into two mirror parts, while longitudinal axis is related to the prosthetic portion and not to the hex connection.

A second improvement of the prior art implant planning software relates the introduction of an algorithm able to make six functions: automatic tracking, overlay, rotation, translation, tilting (inclination) and parallelism.

The “automatic tracking function” traces three landmarks for each digital crown design onto implant, that are the two perpendicular reference planes and the relative vertical axis.

The “overlay function” allows to overlap both of the longitudinal axis of selected abutment on the vertical axis and its plane of symmetry on the one of the two-reference planes thus creating the starting position. Once the abutment is mounted in the starting position, the implant is selected to create an abutment-implant unit. As in the prior art technique, it is necessary to control the bone-to-implant relation after implant's digital placement to obtain the implant axis to be transferred to the custom surgical guide design: an unsatisfactory position will require a well-researched phase of rotation and/or translation and/or tilting and/or creation of parallelism of abutments, that are the four remaining functions of the algorithm.

The “rotation function” around vertical axis allows the counterclockwise rotation of the abutment from the starting position (e.g. 22F of FIG. 9B) in the other three positions through the two-reference planes (e.g. 22M, 22D or 22L of FIG. 9B), thus creating the final position of the abutment.

The “translation function” allows the translation of the starting or final position within their respective digital crown design.

The “tilting function” allows axial tilting of the longitudinal axis from the starting or final position along one or both of two-reference planes.

In the end, in case of digital bridge design, the parallelism function creates the parallelism of longitudinal axes of two abutments, one of which is chosen as the axial reference.

The purpose of the present invention is therefore to overcome the drawbacks of the prior art a implant planning software through the introduction of two perpendicular reference planes (with the relative vertical axis of intersection) in correspondence with digital crown design: faciolingual and mesiodistal, to determine the faciolingual or mesiodistal inclination, respectively, of the straight or angled prefabricated abutment. Intuitively, the faciolingual plane comprises a facial half and a lingual half (respectively 22F and 22L of FIG. 9B) and, similarly, the mesiodistal plane comprises a mesial half and a distal half (respectively 22M and 22D of FIG. 9B). A sign convention is available: rotation, translation or tilting in a facial or mesial direction are positive (+) ; likewise, rotation, translation or tilting in a lingual or distal direction are negative (−). These two reference planes offer the best means of correlating the position of prefabricated abutment with implant position. This means that the prefabricated abutment position should be considered before the implant position. Obviously, since each of the four side faces of any digitally designed crown is usually asymmetric, it should be noted that these two reference planes are to be understood as planes that divide the crown into two halves of equal width both in the faciolingual and in the mesiodistal plane but not as planes of symmetry. In short, this method is easier and less time-consuming for the clinician and makes it easier to control rotation, translation, axial tilting, and parallelism.

A first benefit of the present invention is to provide a pair of reference planes for each digital crown design in order to speed up the determination of the implant insertion axis. In short, this technique lends support to the idea that, performing overlay, rotation or translation of prefabricated abutment on the one of the two-reference planes, implant will follow abutment 3D movement.

This invention allows a second benefit, that is a centered positioning of implant connection within the proposed digital crown.

A third benefit of the present invention is that it allows for parallelism of multiple prefabricated abutments, really helpful in cemented bridges.

Finally, as a fourth benefit, there is the possibility of employing the abutments described by Afflitto Patent U.S. Pat. No. 10,016,259B2 (U.S. patent application Ser. No. 14/632,331 entitled “Inclined abutment for a cemented prosthesis in dental Implantology”) through the algorithm according to the present invention. A unique feature of abutment of Patent U.S. Pat. No. 10,016,259B2 is that the elliptical truncated cone is suitable for correction on both the major longitudinal plane (version 1) and minor longitudinal plane (version 2): in other words, it is the only prefabricated abutment with a double plane of symmetry to be used for correction of implant insertion axis, while any prefabricated abutments available on the market presents a single plane of symmetry. In particular, version 1 of the abutment is suitable for correction of the faciolingually tilted implant of mandibular incisors, canines, premolars and molars. Instead, version 2 of the abutment is suitable for correction of the mesiodistally tilted implant of mandibular incisors, canines, premolars and molars and for correction of the faciolingually tilted implant of maxillary incisors.

### DESCRIPTION OF THE DRAWINGS

FIG. 1A shows a straight prefabricated abutment for cemented prosthesis with its longitudinal axis. FIG. 1B shows an 15° angled prefabricated abutment for cemented prosthesis with its longitudinal axis. FIG. 1C shows an 17° angled prefabricated abutment for screwed prosthesis with its longitudinal axis. FIG. 1D shows a straight prefabricated abutment for screwed prosthesis with its longitudinal axis.

FIG. 2A shows an occlusal view of half mandibular arch. FIGS. 2B-C are detail views of FIG. 2A.

Alignment example of implant hex connection, during implant insertion, for the correction of the facial or lingual implant angulation (FIG. 2B) using the appropriate angled abutment. Alignment example of implant hex connection, during implant insertion, for the correction of the mesial or distal implant angulation (FIG. 2C) using the appropriate angled abutment.

FIG. 3A shows a custom surgical guide (11) with two sleeves (12). FIG. 3B shows a digital implant planning (13) through an implant planning software belonging to the prior art.

FIGS. 4A-C show some sequential steps in the CAD design of custom surgical guide. FIG. 4C shows a CAD design of custom surgical guide before 3D printing. FIGS. 4A-C show digital crown design (14), implant insertion axis (15), digital implant (16) and sleeve (17).

FIG. 5 shows a digital implant planning of two implants. FIG. 5 shows how off-centre placement of the distal prefabricated abutment (18) requires a customized abutment so that the crown has a uniform thickness.

FIG. 6A shows digital crowns design and related digital implants FIG. 6B shows CAD design of customized abutments.

FIG. 7 shows an abutment (19) and an implant (20) with their longitudinal axes.

FIG. 8A-B are a distal perspective views while FIG. 8C is an occlusal view of digital design of a maxillary first right molar crown onto implant (not shown) with the relative faciolingual (21a) and mesiodistal (21b) perpendicular reference planes and vertical axis of intersection (21c).

FIG. 9A shows occlusal view of 10-unit bridge (from maxillary first right molar to first left premolar) retained by 5 implants (each implant crown is marked with four arrows). FIG. 9B shows occlusal view of the two perpendicular reference planes (with the relative vertical axis of intersection 22V) for each implant crown, where, in particular, it is possible to distinguish each of the four directions for correction of the facially tilted implant (22F), for correction of the lingually tilted implant (22L), for correction of the mesially tilted implant (22M) and for correction of the distally tilted implant (22D).

FIG. 10a Shows Version 1 of the Abutment of Patent U.S. Pat. No. 10,016,259B2, which includes major longitudinal plane, corresponding to plane of symmetry (23a), minor longitudinal plane (23b), and angle (α1) that defines the faciolingual correction of abutment respect to the longitudinal axis of the prosthetic portion (23c). FIG. 10B shows version 2 of the abutment of Patent U.S. Pat. No. 10,016,259B2, which includes major longitudinal plane (24a), minor longitudinal plane, corresponding to plane of symmetry (24b), and angle (α2) that defines the mesiodistal correction of abutment with respect to the longitudinal axis of the prosthetic portion (24c).

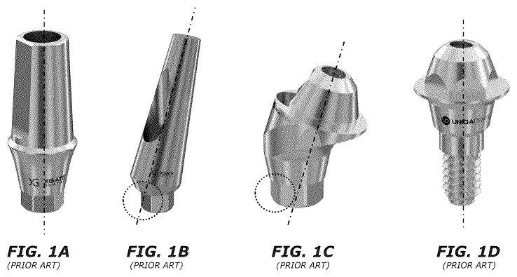

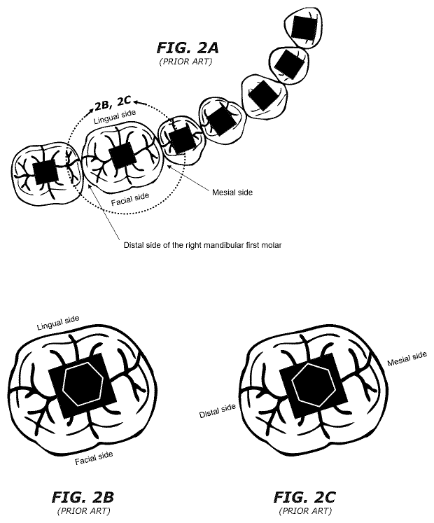

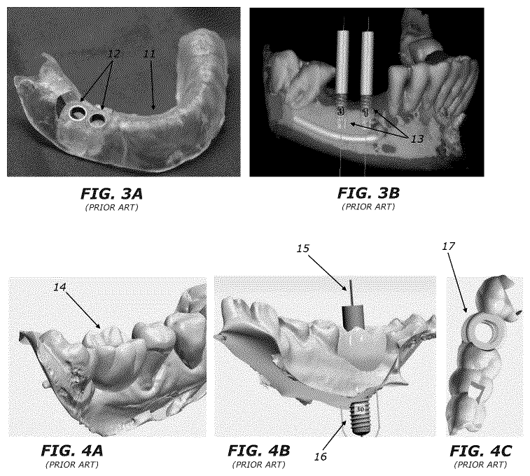

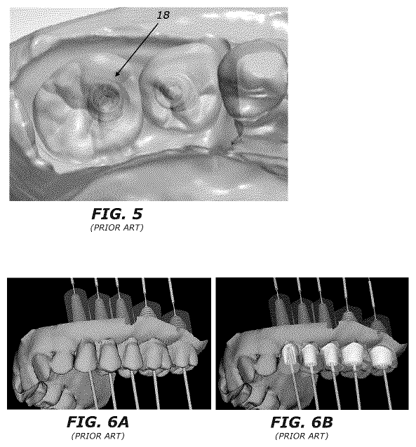

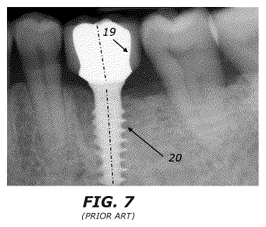

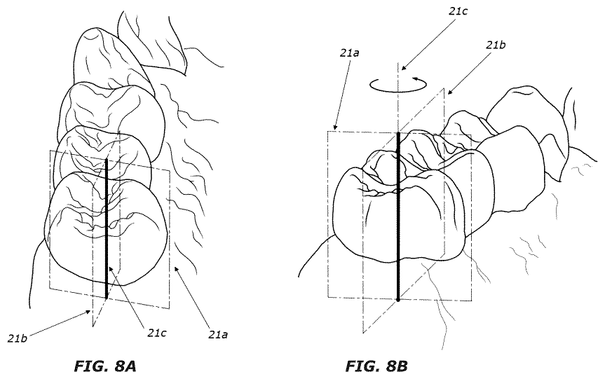

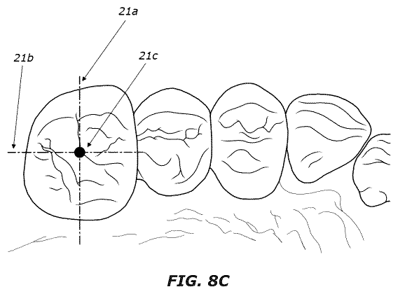

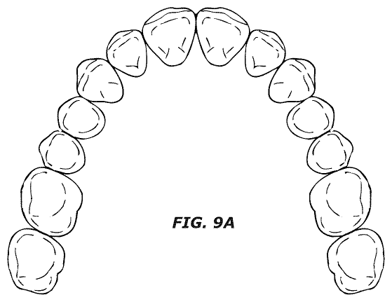

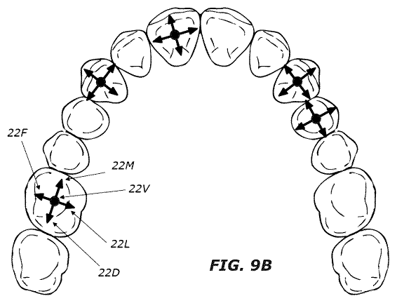

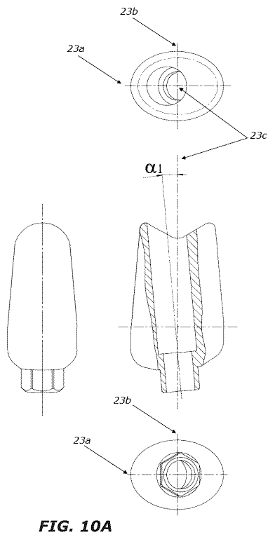

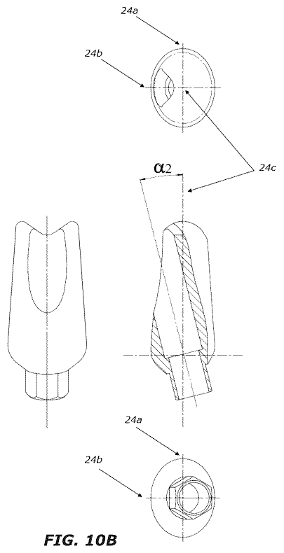

All of the above may be implemented using a display (e.g., computer of mobile phone screen), non-transitory machine-readable storage medium, memory, storage, and/or processor to perform or assist in performing the above-described devices, methods, and steps, and/or may be implemented as part of any of the above described procedures, and/or may be implemented in conjunction with any of the above-described devices.

What is claimed is:

1. A surgical planning computerized method, comprising:
an implant planning software for digital crown design, a modified digital library and an algorithm for the improvement of implant planning software,
wherein said modified digital library contains both the commercially available prefabricated abutments equipped with two landmarks, that are the plane of symmetry and the longitudinal axis, and the commercially available implants, and
wherein said algorithm presents an automatic tracking function to obtain the three landmarks for each digitally designed implant crown, that are the two perpendicular reference planes and the relative vertical axis, and
wherein said algorithm permits an overlay function both of said longitudinal axis on said vertical axis and said plane of symmetry on the one of said two-reference planes, thus creating the starting position of said abutment.

2. The algorithm of claim 1, wherein a rotation function around said vertical axis allows the counterclockwise rotation of said abutment from said starting position in the other three positions through said two-reference planes, thus creating the final position of said abutment.

3. The algorithm of claim 2, wherein a translation function allows the translation of said starting or final position within their respective digital crown design.

4. The algorithm of claim 2, wherein a tilting function allows axial tilting of said longitudinal axis from said starting or final position along one or both of said two-reference planes.

5. The algorithm of claim 2, wherein a parallelism function creates the parallelism of longitudinal axes of two abutments, one of which is chosen as the guide axis.
